(19) United States
(12) Patent Application Publication
(10) Pub. No.: US 2026/0369415 A1
(43) Pub. Date: Nov. 19, 2026

(54) SYSTEMS AND METHODS FOR BUILDING A HEALTHCARE RISK PREDICTION FRAMEWORK USING GATING MECHANISMS

(71) Applicant: NERv Technology Inc., Kitchener (CA)

(72) Inventors: Abdallah Hassen El-Falou, Kitchener (CA); William Michael Kovacs Pitman, Kitchener (CA); Nour Hesham Atif Ahmed Helwa, Ottawa (CA); Raqibul Hayder Chowdhury, Kitchener (CA); Khaled Berry, Kitchener (CA)

(21) Appl. No.: 19/554,233

(22) Filed: Mar. 2, 2026

Related U.S. Application Data
(60) Provisional application No. 63/764,803, filed on Feb. 28, 2025.

(51) Int. Cl. G16H 50/30; G16H 20/00; G16H 50/20

(52) U.S. Cl. CPC G16H 50/30; G16H 20/00; G16H 50/20

(57) ABSTRACT

There is described a system and method for building a healthcare risk prediction framework using gating mechanisms, the method comprising: receiving one or more biomarkers and one or more existing risk prediction models; selecting a set of predictors based on a predetermined threshold, wherein the set of predictors comprises the one or more biomarkers, the one or more existing risk prediction models or a combination thereof; estimating performance of each element in the set of predictors in terms of specificity and sensitivity; combining the set of predictors in a flowchart or a decision tree to generate the healthcare risk prediction framework; calculating a performance estimate of each path in the flowchart or the decision tree; and calculating the performance estimate of the entire flowchart or the entire decision tree.

### CROSS REFERENCE TO RELATED APPLICATIONS

This application claims priority from U.S. Provisional Patent Application No. 63/764,803, filed Feb. 28, 2025, the disclosure of which is hereby incorporated in its entirety by reference.

### FIELD OF THE DISCLOSURE

The present disclosure relates to predicting patient outcomes, in particular, systems and methods for building a healthcare risk prediction framework to predict patient outcomes.

### BACKGROUND

Depending on a patient's medical condition, healthcare providers may recommend performing surgical procedures to improve the patient's quality of life, especially if the condition has already progressed beyond the effectiveness of non-invasive treatments. Surgical procedures may range from being minimally invasive to more complex approaches such as open surgery to treat different pathological conditions and restore body functions. Although modern healthcare has made advancements in developing more effective surgical techniques to improve patient outcomes, there still lies an inherent risk of post-operative complications including, but not limited to, hemorrhages, infections, leakages, ischemia, and sepsis.

One of the most dangerous complications of surgery is a complication known as anastomotic leakage. Anastomotic leakage may develop after an anastomosis is performed where two organs are surgically connected and is most commonly observed in gastrointestinal surgery. Anastomotic leakage leads to luminal contents leaking into the peritoneal cavity which may cause a cascade of deadly complications to arise. This typically involves a form of severe sepsis, peritonitis, morbidity, and it may lead to mortality.

Using traditional techniques, it can take three to seven days on average for an anastomotic leak to be diagnosed since healthcare providers typically wait for symptoms such as abdominal pain, fever, and tachycardia to arise. This is very dangerous, especially considering that every hour of delay causes a considerable increase in the morbidity and mortality risk for the patient.

Leak incidence rate from surgical procedures can vary from 1% to 40% in some cases. Causes behind the development of anastomotic leaks are still being studied with no definitive causes identified yet. There are however risk factors that are associated with high incidence rate such as, but not limited to, age, gender, organ tension, local ischemia, medical history, and surgical errors.

Physiological changes, such as tissue ischemia followed by necrosis, may occur before and during the development of an anastomotic leak. These changes may present themselves as subtle changes in the patient's vital parameters that may not be detected by the current standard of care.

Another serious and potentially severe post-operative complication is known as Postoperative Pancreatic Fistula (POPF), which involves leakage of pancreatic fluid. POPF may occur after performing a pancreaticoduodenectomy, which is more commonly known as a Whipple procedure. This complex surgical procedure is typically performed to treat pancreatic cancer where the pancreatic head is removed along with other parts of the digestive tract which may include the duodenum, gallbladder, and bile duct. Developing a POPF may require complex management of care post-surgery as may be accompanied by abdominal hemorrhage, infection, sepsis, organ failure, and other complications with fatal outcomes.

Each year, 70 million major abdominal surgery (MAS) procedures are performed globally. MAS includes pancreatic, hepatobiliary, and colorectal surgeries with a primary anastomosis. These surgeries have a complication rate of 30-60%, 20% of which have significant detrimental effects that require invasive treatments and enhanced patient monitoring. The occurrence of such complications can have dire consequences, both acute and long-term, including a significant degree of morbidity and mortality for affected patients.

Given the impact of post-operative complications, it is important to ensure that patients are managed appropriately throughout the entire surgical process, from preoperative to postoperative care. Prior to completing surgical procedures, it is especially important to comprehensively evaluate the risks involved in light of possible complications that may arise thereafter. By doing so, healthcare providers can optimize and tailor their approaches early in the surgical process which can lead to improved patient outcomes.

This background information is provided to reveal information believed by the applicant to be of possible relevance. No admission is necessarily intended, nor should it be construed, that any of the preceding information constitutes prior art or forms part of the general common knowledge in the relevant art.

### BRIEF SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which explicitly or implicitly described by the following description and claims.

The object of the present disclosure is a system and method for building a healthcare risk framework using gating mechanisms. Existing methods may be restricted to only allowing for a specific model to be built depending on the type of data that is fed into it. The performance of these models may also be sub-optimal at best in practice. For example, an existing model that evaluates the risk of surgical complications relating to colorectal disorders may have a high false positive rate or low balanced accuracy. As such, there is a need for a method to improve the performance metrics of risk prediction models.

In accordance with an aspect of the disclosure, there is provided a computer-implemented method for building a healthcare risk prediction framework, the method comprising: receiving, at a server through a network, one or more biomarkers of one or more patients and one or more existing risk prediction models; selecting, using a processor, a set of predictors based on a predetermined threshold of the one or more biomarkers or existing risk prediction models, wherein the set of predictors comprises the one or more biomarkers, the one or more existing risk prediction models or a combination thereof; estimating, using the processor, performance of each element in the set of predictors in terms of specificity and sensitivity; combining, using the processor, the set of predictors in a flowchart or a decision tree to generate the healthcare risk prediction framework; calculating, using the processor, a performance estimate of each path in the flowchart or the decision tree; calculating, using the processor, the performance estimate of the entire flowchart or the entire decision tree; determining a risk prediction of one or more patient outcomes based on the flowchart or the decision tree having an optimal performance estimate; and effecting one or more medical procedures to be identified or prioritized based at least in part on the risk prediction, wherein the one or more medical procedures diagnoses, treats or prevents the one or more patient outcomes. In some embodiments, the computer-implemented method further comprises optimizing the combination of the set of predictors in the flowchart or the decision tree using predefined metrics.

In some embodiments, the predefined metrics comprise specificity, sensitivity, accuracy, balanced accuracy, likelihood ratios, predictive values, and F1 scores.

In some embodiments, the performance of each element in the set of predictors is estimated by using experimentation or pooled meta-analysis techniques.

In some embodiments, the combining the set of predictors is based on balancing specificity and sensitivity to minimize false negatives and false positives.

In some embodiments, the flowchart or the decision tree is configured to distinguish cases with a high likelihood of complications and low likelihood of complications.

In some embodiments, the performance estimate of each path in the flowchart or the decision tree is calculated using simulations or probability theory.

In some embodiments, further comprising estimating bounds of expected performance based on dependence between each element in the set of predictors using Fréchet inequalities or simulation techniques.

In some embodiments, the predetermined threshold is determined based on consistent reported data from scientific literature or using simulation techniques.

In some embodiments, wherein the one or more biomarkers comprise at least one of: c-reactive proteins, lipase, amylase, white blood count, procalcitonin, lactate, bilirubin, or neutrophil-lymphocyte ratios.

According to another aspect of the disclosure, there is provided a system for building a healthcare risk prediction framework, the system comprising: a computing device connected to a server through a network, wherein the server comprises: a memory; and a processor coupled to the memory comprising program instructions, wherein the program instructions are executable by the processor to perform operations comprising: receiving, at a server through a network, one or more biomarkers of one or more patients and one or more existing risk prediction models; selecting, using a processor, a set of predictors based on a predetermined threshold of the one or more biomarkers or existing risk prediction models, wherein the set of predictors comprises the one or more biomarkers, the one or more existing risk prediction models or a combination thereof; estimating, using the processor, performance of each element in the set of predictors in terms of specificity and sensitivity; combining, using the processor, the set of predictors in a flowchart or a decision tree to generate the healthcare risk prediction framework; calculating, using the processor, a performance estimate of each path in the flowchart or the decision tree; calculating, using the processor, the performance estimate of the entire flowchart or the entire decision tree; determining a risk prediction of one or more patient outcomes based on the flowchart or the decision tree having an optimal performance estimate; and effecting one or more medical procedures to be identified or prioritized based at least in part on the risk prediction, wherein the one or more medical procedures diagnoses, treats or prevents the one or more patient outcomes.

In some embodiments, the operations further comprise optimizing the combination of the set of predictors in the flowchart or the decision tree using predefined metrics.

In some embodiments, the predefined metrics comprise specificity, sensitivity, accuracy, balanced accuracy, likelihood ratios, predictive values, and F1 scores.

In some embodiments, the performance of each element in the set of predictors is estimated by using experimentation or pooled meta-analysis techniques.

In some embodiments, the combining the set of predictors is based on balancing specificity and sensitivity to minimize false negatives and false positives.

In some embodiments, the flowchart or the decision tree is configured to distinguish cases with a high likelihood of complications and low likelihood of complications.

In some embodiments, the performance estimate of each path in the flowchart or the decision tree is calculated using simulations or probability theory.

In some embodiments, the operations further comprise estimating bounds of expected performance based on dependence between each element in the set of predictors using Fréchet inequalities or simulation techniques.

In some embodiments, the predetermined threshold is determined based on consistent reported data from scientific literature or using simulation techniques.

In some embodiments, the one or more biomarkers comprise at least one of: c-reactive proteins, lipase, amylase, white blood count, procalcitonin, lactate, bilirubin, or neutrophil-lymphocyte ratios.

Other aspects, features and/or advantages will become more apparent upon reading of the following non-restrictive description of specific embodiments thereof, given by way of example only with reference to the accompanying drawings.

### BRIEF DESCRIPTION OF THE DRAWINGS

Several embodiments of the present disclosure will be provided, by way of examples only, with reference to the appended drawings, wherein:

FIG. 1 illustrates an overview of the process to build a healthcare risk prediction framework using gating mechanisms, in accordance with one embodiment.

FIG. 2 illustrates a visual representation in which an exemplary calculation of biomarkers in terms of sensitivity is based on, in accordance with one embodiment.

FIG. 3 illustrates a visual representation in which an exemplary calculation of biomarkers in terms of specificity is based on, in accordance with one embodiment.

FIG. 4A illustrates a visual depiction of the determination of lower bounds on the intersection of events, in accordance with one embodiment.

FIG. 4B illustrates a visual depiction of the determination of upper bounds on the intersection of events, in accordance with one embodiment.

FIG. 5A illustrates a scenario in which a rule-out gate with negative test is used in a model, in accordance with one embodiment.

FIG. 5B illustrates a scenario in which a rule-in gate with positive test is used in a model, in accordance with one embodiment.

FIG. 5C illustrates a scenario in which a rule-in and rule-out gate with positive and negative test is used in a model, in accordance with one embodiment.

FIG. 6 is a flowchart showing one possible way of implementing biomarker tests in a new risk prediction framework, in accordance with one embodiment.

FIG. 7 is a flowchart showing another possible way of implementing biomarker tests in a new risk prediction framework, in accordance with one embodiment.

FIG. 8A illustrates an exemplary POD 1 decision tree, in accordance with one embodiment.

FIG. 8B illustrates an exemplary POD 2 decision tree, in accordance with one embodiment.

FIG. 8C illustrates an exemplary POD 3 decision tree, in accordance with one embodiment.

FIG. 8D illustrates an exemplary POD 5 decision tree, in accordance with one embodiment.

FIG. 9A is a table that outlines groupings applied to biomarker gates, in accordance with one embodiment.

FIG. 9B illustrates an exemplary gating flowchart based on groupings of biomarker gates, in accordance with one embodiment.

FIG. 10A is a table that outlines theoretical biomarker metrics for different scenarios in relation to various PODs, in accordance with one embodiment.

FIG. 10B is a table that outlines theoretical biomarker metrics in relation to post-operative day 3, in accordance with one embodiment.

FIG. 11A illustrates a workflow that may be implemented using gates based on POD 1 amylase and POD 2 CRP, in accordance with one embodiment.

FIG. 11B illustrates a workflow that may be implemented using gates initially based on POD 1 amylase and POD 2 CRP after excluding expired tests, in accordance with one embodiment.

FIG. 11C illustrates a workflow that may be implemented using replacement gates to gates that have expired, in accordance with one embodiment.

FIG. 12A is a table outlining groupings applied to biomarker gates specifically associated with a lipase center, in accordance with one embodiment.

FIG. 12B is a table outlining groupings applied to biomarker gates specifically associated with an amylase center, in accordance with one embodiment.

FIG. 13A illustrates a graphical representation of PODs in which lipase tests are applied to a risk prediction framework, in accordance with one embodiment.

FIG. 13B illustrates a graphical representation of PODs in which amylase tests are applied to a risk prediction framework, in accordance with one embodiment.

FIG. 14 is a schematic diagram of a computing system used to predict one or more patient outcomes based on risk prediction models using gating mechanisms, in accordance with one embodiment.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

When introducing elements of aspects of the disclosure of the examples thereof, the articles “a,”, “an,”, “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

The terms “biomarker(s)” as used herein may refer to molecules, substances, and chemical or physical properties that can be measured or detected as bio-signals in bodily fluids. They include, but are not limited to, c-reactive proteins (CRP), lipase, amylase, white blood count, procalcitonin, lactate, bilirubin, neutrophil-lymphocyte ratios, pH, temperature, electrolyte concentration, fluid flow rate, pressure, lactic acid, nitrates, alkali ions, inflammatory proteins, bacterial proteins, specific cells, molecules, genes, gene products, enzymes, hormones, inflammatory proteins, and glucose.

It is an object of the present disclosure to provide systems and methods for building a healthcare risk prediction model using gating mechanisms to predict one or more patient outcomes, including but not limited to post-operative complications. Post-operative complications, such as anastomotic leakage, can be divided into different classifications based on certain criteria including, but not limited to, severity of the complication and type of recommended treatment pathway. One such classification is a standardized definition for anastomotic leaks established by the International Study Group of Rectal Cancer (ISREC) in 2010. The definition classifies anastomotic leaks into three different grades—grade A, grade B, and grade C—based on the impact on clinical management. Grade A anastomotic leakage results in no change in management of a patient that has no requirement of active therapeutic intervention. Grade B anastomotic leakage requires active therapeutic intervention and does not require reoperation such as a relaparotomy. Grade C anastomotic leakage requires reoperation. Based on the aforementioned definition, grade A anastomotic leakage is categorized as a non-clinically relevant leak, while grade B and C anastomotic leakage are categorized as clinically relevant leaks as these more significantly impacts a patient's medical condition.

A similar classification system also applies to POPF established by the International Study Group of Pancreatic Surgery (ISGPS), wherein grade B and C are categorized as clinically relevant, requiring one or more interventions as a result of a patient's condition after pancreatic surgery. Grade A POPF, as originally established, no longer exists as of 2016 and has been replaced by a category known as a biochemical leak which is characterized by an asymptomatic pancreatic leak. Nevertheless, these classifications are especially important considerations when with respect to predicting risk of post-operative complications.

The terms “NO”, “NOA”, “BC”, “ABC” as used herein refer to uncomplicated non-leaks, other complications, clinical leaks (grade B or grade C), and all leaks (grade A, grade B, grade C), respectively. The terms “POD” and “POH” as used herein refers to post-operative day and post-operative hour, respectively. In addition, the term “ALRC” refers to anastomotic leak risk classification model(s).

The term “flowchart” as described herein refers to a representation of a general workflow or process in a sequential manner. The term “decision tree” as described herein is a representation of possible outcomes from a series of decisions based on a set of conditions.

Systems and methods disclosed herein may be utilized to build optimal and improved healthcare risk prediction frameworks by using at least previously existing prediction models. Embodiments of the optimal and improved prediction framework may rely on having gates that are either strong in sensitivity and weak in specificity, or strong in specificity and weak in sensitivity to more accurately predict whether a patient has a high or low risk of adverse outcomes. This allows for incorporation of existing biomarker tests into existing prediction frameworks with or without data of how the biomarkers interact with the existing prediction frameworks and allows for a plurality of risk prediction models to be chained together into one or more entirely new risk prediction frameworks. In the case when data is unavailable in terms of how the biomarkers interact, sound assumptions are made as they are incorporated into existing prediction frameworks. An example of an assumption that may be made is that the biomarkers are independent (i.e., the outcome of one biomarker does not impact the outcome of another biomarker).

Gates described herein comprise exclusionary and inclusionary criteria in classification models. These criteria are associated with different biomarkers, wherein the biomarkers and their corresponding criteria are contextualized based at least on a type of medical specialty, type of procedure, and/or type of surgery (i.e., elective or emergent surgery, open surgery or minimally invasive surgery). Other considerations may be considered including normal ranges of biomarker values after surgery and patient factors such as obesity and age. For example, lipase or amylase may be used as the basis of the gates in the model when it comes to evaluating the risks of anastomotic leaks in Hepato-Pancreato-Biliary (HPB) procedures. Along with lipase or amylase, CRP may also be used as the basis of gates in the model when it comes to evaluating the risks of post-operative pancreatic fistula (POPF) in patients undergoing pancreatic surgery.

In the case when there is a lack of source data to build models around certain biomarkers, it may be possible to conduct a risk assessment by using one or more gates. The relationships with existing models in terms of expected performance may be taken into account in refining the overall risk prediction framework in such a way that is complementary to the chosen gates. For example, an AND gate with a high sensitivity biomarker may be more likely to be complementary with a high sensitivity classifier since the overall sensitivity drop will be small while the overall specificity rise will be large. There may also be an enhancement in the ability of a model to predict risk by specifically choosing gates that are heavily weighted towards sensitivity and/or specificity relative to accuracy metrics.

The gating mechanisms underlying the disclosed risk prediction frameworks are applicable to any risk assessment or predictive process in healthcare, including but not limited to diagnostics, screening, and disease prediction. However, for purposes of illustration of an exemplary embodiment, risk prediction frameworks for post-operative complications are primarily described herein.

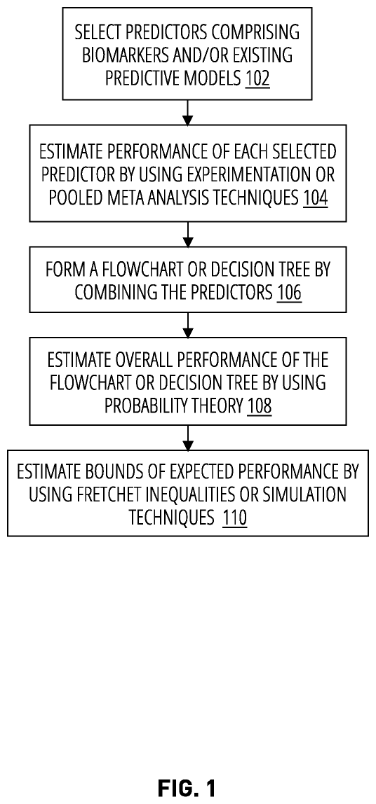

FIG. 1 illustrates an overview of the process to build a healthcare risk prediction model using gating mechanisms. A set of predictors comprising biomarkers and/or existing predictive models are selected in step 102, wherein each predictor has a predetermined threshold. If a predictor is a biomarker such as amylase, a threshold may be 300 U/L. If a predictor is an existing machine learning model, a threshold may be a probability output of 55%. Performance estimates of each predictor in predicting the outcome of patients are obtained in step 104 by either using experiments if data for the predictors and patients are available or using literature if data is not available. Multiple sources that use pooled meta-analysis techniques may be used to estimate the performance of the predictors at a given threshold or cutoff value. These sources may also be a used to estimate the confidence intervals for the performance of the predictors. Once performance estimates of each predictor are determined, flowcharts or decision trees utilizing the predictors are formed in step 106. For example, given predictor A, B and C, flowchart decisions may be: if predictor A is high, predict high risk, if low, pass forward; next, if predictor B is low, predict low risk, if high, pass forward; last, if predictor C is high, predict high risk, if low, predict low. When funneling a patient through the flowchart or decision tree based on the predictors, the estimated sensitivity or specificity of the predictors should minimize false positives and false negatives. Once a flowchart or decision tree is formed, performance estimates of each path in the flowchart or decision tree as well as performance estimates of the entire flowchart or decision tree are determined in step 108. These performance estimates are determined using probability theory, wherein each path has a conditional probability to be calculated. For example, based on the aforementioned example, one path in the flowchart may be predictor A is low, and predictor B is low, resulting in a low risk prediction. The estimated probability of a patient going down this particular path can be calculated in terms of when the patient will or will not have the outcome being predicted using the theoretical sensitivity and/or specificity of each test. Simulation techniques may also be used to determine the overall performance estimate of the flowchart or decision tree. Estimated bounds of expected performance are then determined using Fréchet inequalities or simulation techniques in step 110. Fréchet inequalities may be used to estimate the bounds of expected performance based on the dependence of each predictor compared to one another. This estimation can be performed for each path in the flowchart to determine an overall sensitivity and specificity of the predictors utilized together. Conversely, simulated datasets may be used to achieve performance estimation. If datasets of sufficient size are simulated with performance of each predictor, then estimates of different methods of linking together predictors can be calculated. The dependence of the predictors may also be incorporated into the dataset if there are estimates of these as well. In addition, simulated datasets may be used to calculate optimal methods to combine predictors together if a given metric to optimize for, such as balanced accuracy, can be defined.

In general, the thresholds or cutoff values in relation to different biomarkers are predetermined based on those used or reliably reported in literature. If there is no consensus to thresholds in literature for a given clinical factor, then optimal thresholds may be determined by conducting simulations or experiments on different thresholds and validating their performance. The latter allows for more flexibility to determine optimal thresholds, which may be beneficial when it comes to building a risk prediction framework based on existing models and biomarkers.

In some embodiments, the method of building decision flowcharts or decision tree may produce an optimal overall performance by incorporating multiple biomarkers that either have a high sensitivity or high specificity, even if the counterpart metric is not high (e.g., low specificity when sensitivity is high). This is because patients can move forward to high or low prediction decisions by these biomarkers with more confidence. As such, patients associated with less confidence are passed forward to additional tests in the flowchart or decision tree.

A general exemplary scenario is provided herein to describe how a biomarker may be chosen as a gate to a post-operative risk prediction model. Consider a biomarker that is 100% sensitive but 50% specific on the first post-operative day of a patient. This biomarker is chosen as a gate in the model since it will accurately predict every occurrence of a post-operative complication with no performance deterioration but may also report false positives. In this case, if the biomarker predicts the absence of a post-operative complication, the further gates in the model are bypassed and a low-risk output is determined. Conversely, if the biomarker predicts the presence of a post-operative complication, the process of predicting risk using the model continues to the next gates, if applicable. A risk level output is then determined at each subsequent gate in the model. In this example, there is no risk in implementing such a gate since it does not overlook patients that may not have any risk of post-operative complications. However, there may be cases wherein the model may report high risk, in which case the biomarker may circumvent a false positive.

Given that there are typically no biomarkers that exist which are 100% sensitive or 100% specific, the main factors that affect whether a biomarker is sufficient to be implemented as a gate are the ability of the biomarker to assist in making overall risk predictions and the ease in which the biomarker can be obtained from the field. Selecting the gates also involves ensuring that an optimal performance is achieved for a given model, specifically the performance of a post-operative risk model such as an anastomotic leak risk model in the case of the present disclosure. Combining a gate with high sensitivity may be unlikely to significantly affect sensitivity but may significantly increase specificity. In a similar manner, a gate with high specificity may be unlikely to significantly affect specificity but may significantly increase sensitivity.

An exemplary scenario is provided below to describe the effect of combining biomarker criteria. Assuming two biomarkers ABC and XYZ are independent and uncorrelated, probabilities may propagate as is standard (i.e., multiplication for logical ‘and’, addition for logical ‘or’). In the case that high-sensitivity criteria are added, the two criteria are combined with a logical ‘and’ operation wherein the formal condition is if ABC is true and XYZ is true, risk is high; otherwise, risk is low. Referring to Table 1, assuming independence of biomarkers ABC and XYZ, it is expected that 90% and 91% of post-operative complications are true positives in relation to biomarker ABC and XYZ, respectively. As such, the sensitivity associated with ABC ‘and’ XYZ is 82%. False negative rates are calculated by the inverse of the sensitivity values (i.e., subtract the corresponding sensitivity values from 1). The same logic may be applied to the other performance metrics of the model. In terms of specificity, it is determined based on the corresponding false positive rates since the false positive rates correspond with positive predictions on negative labelled patients. In particular, specificity values are calculated by the inverse of the false positive rate values (i.e., subtract the corresponding false positive rate value from 1 to determine specificity). Generally, a plurality of sensitivity and false positive rate values are required since an ‘and’ operation is performed on positive predictions from the model in this scenario. As a result, there is an improvement in the balanced accuracy associated with incorporating both biomarkers ABC and XYZ as gates in a given risk prediction model compared to if the model only relied on either one of the two biomarkers.

TABLE 1
Post-Operative Risk Model Performance Based on an
Exemplary Set of Biomarkers. Each performance metric
in the table is expressed in percentages.

| | | | False | False | |
|---|---|---|---|---|---|
| | | | Negative | Positive | Balanced |
| | | | Rate | Rate | Accuracy |
| Biomarker | Sensitivity | Specificity | (FNR) | (FPR) | (BA) |
| ABC | 90 | 70 | 10 | 30 | 80 |
| XYZ | 91 | 60 | 9 | 40 | 75 |
| ABC and XYZ | 82 | 88 | 18 | 12 | 85 |

In some embodiments, the basis of the gating mechanisms in the post-operative risk prediction framework are based on using tests that are very strong in sensitivity and weak in specificity or very strong in specificity and weak in sensitivity. This would allow patients to be gated to either positive or negative predictions. Table 2 illustrates an example of biomarker tests, ABC and XYZ, with strong sensitivity and weak specificity given that the tests are independent (e.g., a positive or negative reading from one test does not influence whether the other test will be positive or negative). These may potentially be used in a complimentary method that improves one metric while minimizing the loss in another metric. This can be performed by taking an ‘and’ of the two tests (e.g., test ABC and XYZ need to both be positive for a patient to be overall positive).

TABLE 2
Post-Operative Risk Model Performance Based
on Another Exemplary Set of Biomarkers.

| | Sensitivity | Specificity | Balanced |
|---|---|---|---|
| Biomarker | (%) | (%) | Accuracy (%) |
| ABC | 90 | 60 | 75 |
| XYZ | 89 | 50 | 69.5 |
| ABC and XYZ | 80 | 80 | 80 |

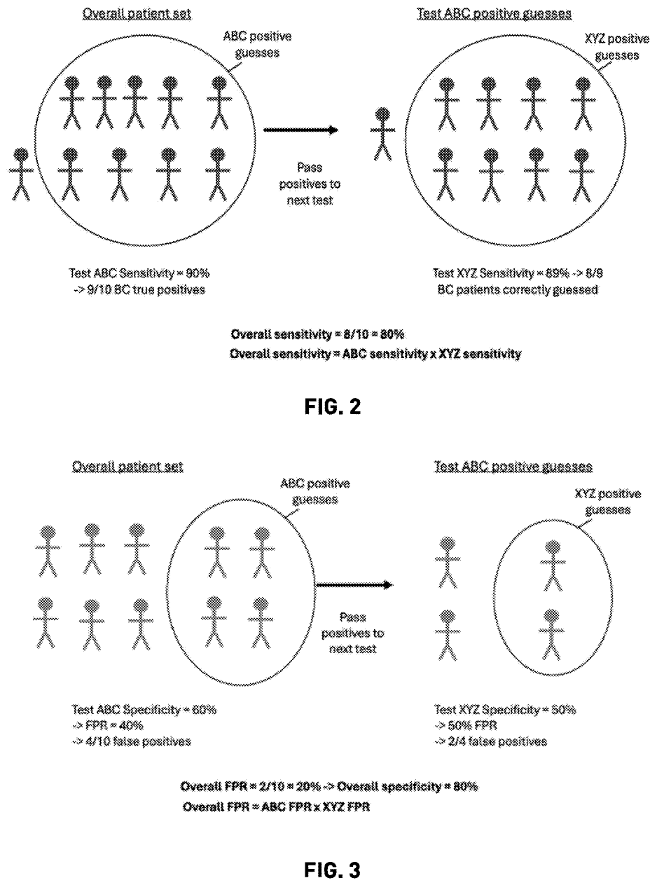

FIG. 2 illustrates a visual representation in which the calculation of ABC ‘and’ XYZ in terms of sensitivity is based on as presented in the Table 2. As shown in the figure, there are 10 patients who have a grade B or C post-operative complication. In this case, sensitivity is visually shown as the percentage of these patients that are true positives, wherein sensitivity (or the percentage of patients who have a grade B or C post-operative complication) should be the same on two subsets of patients assuming independence of tests. A test is performed on the overall patient set to visually show the sensitivity of the ABC biomarker in which 90% of the patients were true positives. Another test is also performed on the subset of patients who were true positives based on the ABC test to visually show the sensitivity of the XYZ biomarker in which 89% were found to be true positives. Multiplying the two measures of sensitivity yields an overall sensitivity of 80%, which is generally lower than each of the individual tests combined. The lower sensitivity value is a result of requiring both tests to be correct for a positive diagnosis.

FIG. 3 illustrates a visual representation in which the calculation of ABC ‘and’ XYZ in terms of specificity is based on as presented in the Table 2. As shown in the figure, there are 10 patients that have a non-clinically relevant post-operative complication, which may include grade A post-operative complication. In this case, specificity is determined on the basis of first measuring the percentage of these patients that are false positives. A test is performed on the overall patient set to visually show the specificity of the ABC biomarker in which 40% of the patients were false positives, indicating that the specificity value is 60%. In a similar cascading manner as that of FIG. 2, the subset of determined patients were passed to the next test to visually show specificity in terms of the XYZ biomarker in which 50% were false positives. The overall measure of specificity is determined to be 80% on the basis of the overall false positive rate between the false positive percentages from the ABC and XYZ tests, which is determined multiplying the two false positive measures which yields 20%.

Overall, the method to determine the overall specificity associated with using both biomarker tests, if the tests are independent, generally mirrors the method to determine overall sensitivity. An ‘and’ operation is used on positive predictions from each test to determine an overall calculation, which also represents P(A∩B). Given that the focus is to evaluate the positive predictions, false positive rate values are used instead of specificity. The same cascade of calculations is performed, but the differences is that false positives are examined rather than true positives. Based on Table 2, the cascading operation causes a cost to overall sensitivity. However, the drop in sensitivity is lower than the rise of specificity. This also applies to the set of exemplary data shown in Table 1.

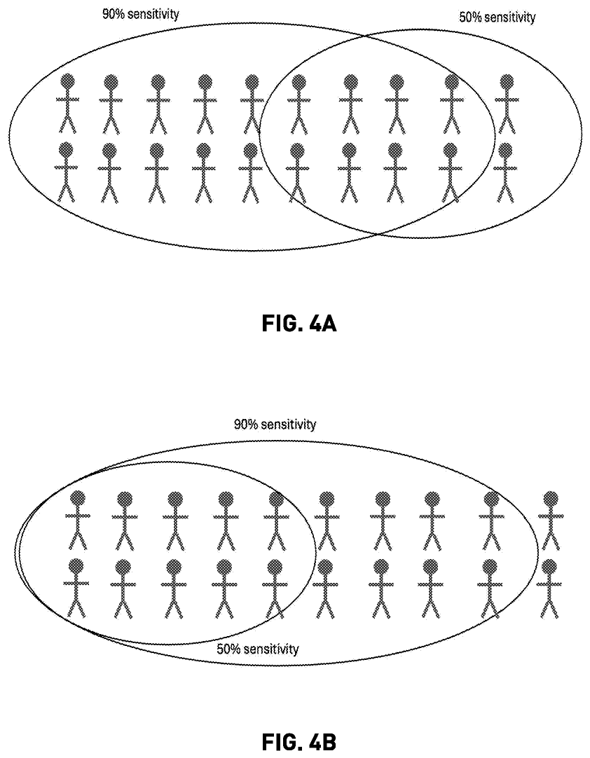

In the case of tests that are not independent, the measurement of sensitivity and specificity depends on the use of Fréchet inequalities to calculate the upper and lower bounds on the intersection of events of positive outcomes. This is represented by the following probability expression: max(0, P(A)+P(B)−1)≤P(A∩B)≤min(P(A), P(B)). FIG. 4A and FIG. 4B illustrate a visual depiction of the determination of lower bounds and upper bounds, respectively, on the intersection of events. Given a scenario with 20 patients and two tests having 90% and 50% sensitivity, the lower bound shown in FIG. 4A is determined to cover 40% of the patients while the upper bound shown in FIG. 4B covers 50% of the patients. These upper and lower bounds may be used in relation to previously aggregated metrics shown in Table 2 (shown in Table 3).

TABLE 3
Post-Operative Risk Model Performance Based on Another Exemplary
Set of Biomarkers with Upper and Lower Bounds.

| | Sensitivity | Specificity | Balanced |
|---|---|---|---|
| Biomarker | (%) | (%) | Accuracy (%) |
| ABC | 90 | 60 | 75 |
| XYZ | 89 | 50 | 69.5 |
| ABC and XYZ | 80 [79-89] | 80 [60-100] | 80 [69.5-94.5] |

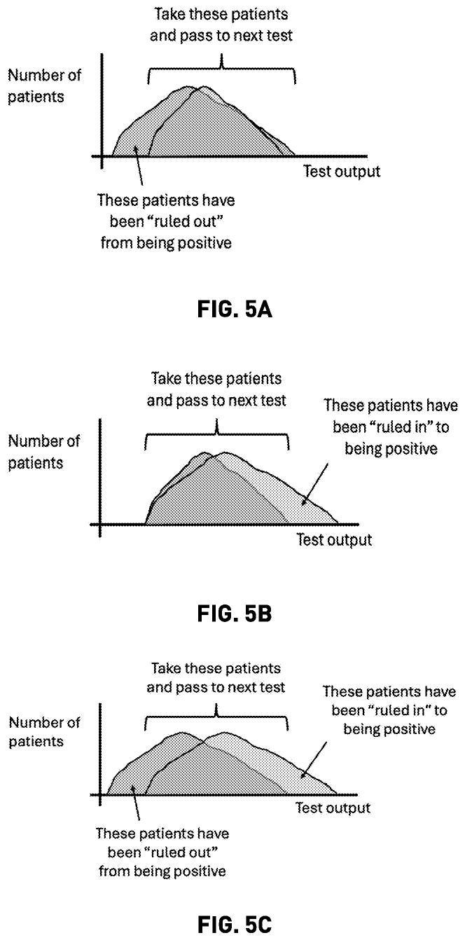

When it comes to the application of gating mechanisms in the present disclosure, a test that operates at an extreme to improve performance of another test may be utilized. As shown in FIG. 5A-5C, there may be different scenarios in which a screening test may be used to rule in or rule out patients in a model, including a rule-out gate with negative test (FIG. 5A), a rule-in gate with positive test (FIG. 5B), and a rule-in and rule-out date with positive and negative test (FIG. 5C). For instance, having two very high sensitivities but weak specificities increase the overall balanced accuracy since sensitivity drops less than the extent of the rise in specificity. The same technique may be used with tests that have high specificity but low sensitivity, wherein taking an ‘and’ on negative predictions would be completed instead of taking an ‘and’ on positive predictions.

The present disclosure comprises the use of pooled meta-analysis based on different studies to determine an expected performance of different metrics such as sensitivity and specificity at a given threshold in relation to various biomarkers. Pooled meta-analysis may be based on previous meta-analysis studies available in literature or performed by a user building the risk prediction model. Generally, sensitivity and specificity values are not pooled over all thresholds since the variations will skew the estimations. To avoid this, similar thresholds and patient sets may be grouped (e.g., tests that used a cutoff value of 900-1000 U/L) and performance around this threshold can be estimated. A fixed or random effects model may be used as strategies to estimate an overall sensitivity or specificity performance after grouping metrics from a similar threshold (e.g., around 1000 U/L).

A fixed effects model assumes that the true test accuracy is the same across all studies (e.g., when using amylase with a threshold of 1000 U/I, the sensitivity is a value of X across all studies, and variation across studies is due to random sampling). Calculation of sensitivity and specificity may be completed by using a weighted average that takes into account either the study size or the study size and the proportion of the complication such as an anastomotic leak.

Conversely, a random effects model assumes that the different studies have different magnitudes of test accuracy (e.g., when using amylase with a threshold of >1000 U/I, the sensitivity is a value of X for study A, Y for study B, and so on). The difference in values for each study is attributed to differences in study populations or procedures used. The calculation of sensitivity and specificity may be completed using a weighted averages that take the same factors as the fixed model into account, along with an estimate of variation between studies. Random effects models may be typically used, since there is almost always some presumed difference between studies in systemic reviews.

Another strategy to determine an estimate of model performance is using a Summary Receiver Operating Characteristic (SROC) curve, which involves the utilization of various sensitivities and specificities reported in different studies. Although this method provides an overall estimate of the performance of a test over various thresholds or cutoff values, it does not examine how a test may perform at a given threshold. As such, pooled metric calculations are used to determine the latter.

In terms of investigation strategies to determine an estimate of model performance, including pooled metric calculations and SROC, heterogeneity needs to be accounted for. This involves investigation into whether changes in performance between studies are larger than expected due to confounding factors, and whether further grouping or exclusion is warranted. Some of the sources of variation that need to be taken into account may comprise type of surgical approaches (e.g., Whipple, pancreatectomy), type of post-surgical complication (i.e., clinically relevant vs. non-clinically relevant anastomotic leaks), different post-operative measurement times of biomarkers, and different thresholds used for classification.

In order to build a post-operative risk prediction framework using a plurality of gates, there are different factors that need to be taken into account which may comprise different methods of combining gates. A set of test data, each characterized by different performance metrics, is used herein to demonstrate how equations can be built to determine different performance metrics such as sensitivity and specificity in relation to the gates. This test data is found in Table 4, wherein there are three exemplary tests described in terms of their performance metrics. In this case, all three tests have a balanced accuracy of 70%. Test A optimizes specificity, test B optimizes sensitivity, while test C is balanced. With the use of gating, the test data is demonstrated to improve the overall balanced accuracy which will be described hereafter. The order of gates in a model also impacts whether sensitivity or specificity is optimized.

TABLE 4
Exemplary Performance Metrics for Different Gates. Each performance
metric in the table is expressed in percentages.

| | | | | False | False |
|---|---|---|---|---|---|
| | Balanced | | | Negative | Positive |
| | Accuracy | Sensitivity | Specificity | Rate | Rate |
| Test A | 70 | 50 | 90 | 50 | 10 |
| Test B | 70 | 90 | 50 | 10 | 50 |
| Test C | 70 | 70 | 70 | 30 | 30 |

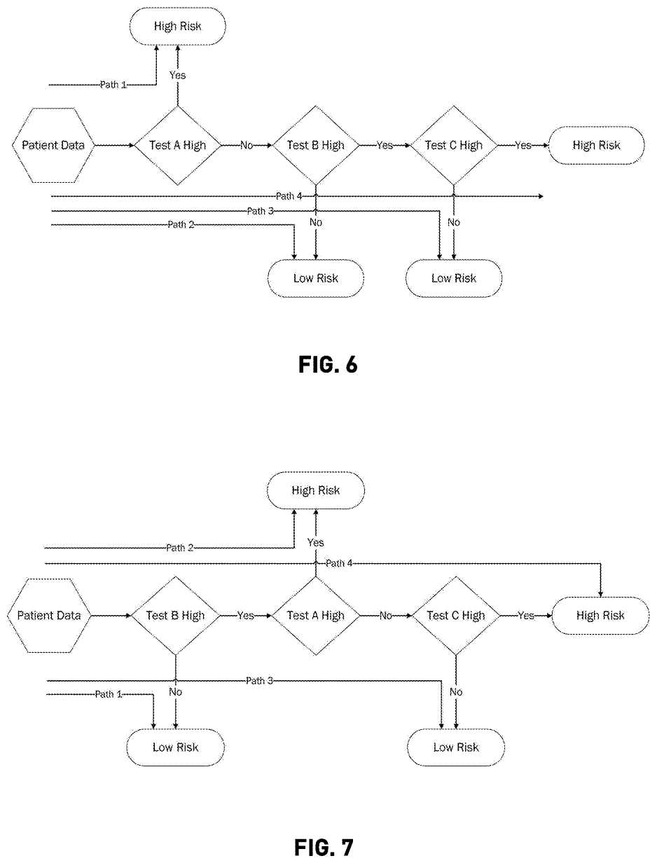

FIG. 6 is a flowchart showing one possible way of implementing the tests in Table 4. There are different paths outlined in the flowchart to illustrate the possible chain of events to determine an overall probability of an event or prediction of high risk or low risk. Path 1 leads to a prediction of high risk after going through one gate in the model. Path 2 leads to a prediction of low risk after going through two gates in the model. Path 3 and path 4 lead to a prediction of low risk and high risk, respectively, after going through 3 gates in the model. Each of paths 1-4 are mutually exclusive since a patient cannot be at the endpoint of multiple paths. To calculate the overall probability of an event and determine a prediction of risk, the probability of each path needs to be determined. Based on FIG. 6, the probability equations for each path are as follows:
P(Path1)=P(A_{high})
P(Path2)=P(A_{low}∩B_{low})
P(Path3)=P(A_{low}∩B_{high}∩C_{low})
P(Path4)=P(A_{low}∩B_{high}∩C_{high})

To calculate overall sensitivity and specificity, the probabilities of each of the paths need to be considered. The overall sensitivity is the probability of going down either Path 1 or Path 4, given a BC patient. The overall specificity is the probability of going down Path 2 or Path 3, given a NOA patient. These equations become as follows:
Sensitivity=P(High|BC)=P(Path1∪Path4|BC)=P(A_{high}∪(A_{low}∩B_{high}∩C_{high})|BC)
Specificity=P(Low|NOA)=P(Path2∪Path3|NOA)=P((A_{low}∩B_{low})∪(A_{low}∩B_{high} ∩C_{low})|NOA)

Regarding the calculation of unions, the probabilities are added since the paths are mutually exclusive (i.e., no patient could go down both Path 2 and Path 3). As for the calculation of intersections, independence is assumed for simplicity of the current provided example, so the probabilities are multiplied. However, there may be some dependence in reality. Limits may be calculated for more formalized gating scenarios. Given the overall sensitivity and specificity equations as described above, the new metrics are determined by substituting the metrics as described in Table 4. The calculations of overall sensitivity and specificity are as follows:
Sensitivity=P(A_{high}∪(A_{low}∩B_{high}∩C_{high})|BC)=0.5+(0.5×0.9×0.7)=0.815
Specificity=P((A_{low}∩B_{low})∪(A_{low}∩B_{high}∩C_{low})|NOA)=(0.9×0.5)+(0.9×0.5×0.7)=0.765.

By taking the average of the calculated sensitivity and specificity values (81.5% and 76.5%), the theoretical balanced accuracy is determined to be 79%. This value improves the previous balanced accuracy value of 70% outlined in Table 4 for each of the individual tests. In order to validate the calculation of overall sensitivity and specificity, calculations for false negative rate and false positive rate are determined. The false negative rate should reflect a value that is the sensitivity value subtracted by 1, while the false positive rate should reflect a value that is the specificity value subtracted by 1. These values are calculated as follows:
False Negative Rate=P((A_{low}∩B_{low})═(A_{low}∩B_{high}∩C_{low})|BC)=(0.5×0.1)+(0.5×0.9×0.3)=0.185
False Positive Rate=P(A_{high}∪(A_{low}∩B_{high}∩C_{high})|NOA)=0.1+(0.9×0.5×0.3)=0.235

Furthermore, the order of gates is an important consideration when building the risk prediction framework with these gating mechanisms. FIG. 7 is a flowchart showing another possible way of implementing the tests in Table 4, wherein test A and test B are swapped in order compared to FIG. 6. Based on FIG. 7, the probability equations for each path are as follows:
P(Path1)=P(B_{low})
P(Path2)=P(B_{high}∩A_{high})
P(Path3)=P(B_{high}∩A_{low}∩C_{low})
P(Path4)=P(B_{high}∩A_{low}∩C_{high})

Similarly, the probabilities of each of the paths need to be considered to calculate overall sensitivity and specificity. The overall sensitivity is the probability of going down either Path 2 or Path 4, given a BC patient. The overall specificity is the probability of going down Path 1 or Path 3, given a NOA patient. These equations and the corresponding calculation of sensitivity and specificity based on the values in Table 4 are as follows:
Sensitivity=P(High|BC)=P(Path2∪Path4|BC)=P((B_{high}∩A_{high})∪(B_{high}∩A_{low}∩C_{high})|BC)=(0.9×0.5)+(0.9×0.5×0.7)=0.765
Specificity=P(Low|NOA)=P(Path1∪Path3|NOA)=P(B_{low}∪(B_{high}∩A_{low}∩C_{low})|NOA)=0.5+(0.5×0.9×0.7)=0.815

By swapping gating test A and test B in the model, the specificity has become higher than sensitivity. Overall, if test A is set as the first gate, all predictions are funneled to high before going through other tests, which causes the optimization for high predictions (sensitivity). Conversely, if test B is set as the first gate, all predictions are funneled to low before going through other tests, which causes the optimization for low predictions (specificity).

Beyond ordering, additional methods may be used to combine tests together in a prediction model. For example, tests may be grouped, and a rule may be “if any of these tests are high, overall test is high” or “if all these tests are high, overall test is high”. Doing so groups tests and specifies an ‘or’ or ‘and’ operation between them.

TABLE 5
Exemplary Performance Metrics to
Determine a Combination of Tests.

| | Sensitivity | Specificity | False Negative | False Positive |
|---|---|---|---|---|
| | (%) | (%) | Rate (%) | Rate (%) |
| Test A | 90 | 50 | 20 | 40 |
| Test B | 90 | 50 | 20 | 40 |

Given a set of data as shown in Table 5, a new test C may be determined which has the following rule: “if test A or test B is high, predict high”. This rule implies that there is a union between test A and test B. Given this rule, theoretical values of sensitivity and specificity for test C, assuming that tests A and B are independent, are calculated as follows:
Sensitivity=P(High|BC)=P(A_{high}∪B_{high}|BC)=P(A_{high}|BC)+P(B_{high}|BC)−(A_{high}∩B_{high})|BC)=0.9+0.9−(0.9×0.9)=0.99
Specificity=P(Low|NOA)=P(A_{low}∩B_{low}|NOA)=0.5×0.5=0.25

If the tests A and B are dependent, Fréchet inequalities are used to determine the theoretical bounds. By using this, the bounds relating to sensitivity would be between 0.9 and 1, while the bounds relating to specificity would be between 0 and 0.5.
Sensitivity: max(P(A), P(B))≤P(A∪B)≤min(P(A)+P(B), 1)
Specificity: max(0, P(A), P(B)−1)≤P(A∪B)≤min(P(A), P(B))

By performing an ‘or’ operation on two tests, a very high theoretical sensitivity is generated, but at the cost of specificity. The use of these grouping rules as a technique to optimize performance may potentially be used to construct new gates from biomarkers with less extreme metrics (e.g. two biomarkers with sensitivity lower than 90%).

Decision trees are used to examine optimal gating strategies. Synthetic datasets are constructed as a basis of a simulation to determine an optimal application of biomarkers as gates in a risk prediction model. The constructed synthetic datasets are constrained so that expected biomarker sensitivity and specificity values are attained. As an exemplary scenario, the following conditions are used for a simulation:
50,000 patients
25% leak rate
Biomarkers are provided as flags, e.g. if POD 3 CRP>122 has sensitivity of 91% and specificity of 41%, then 91% of BC patients have a value of 1 for POD 3 CRP>122 and 41% of NOA patients have a value of 0 for POD 3 CRP>122
A patient with low amylase cannot have high amylase and patient with low lipase cannot have high amylase
Remaining biomarkers are independent from one another

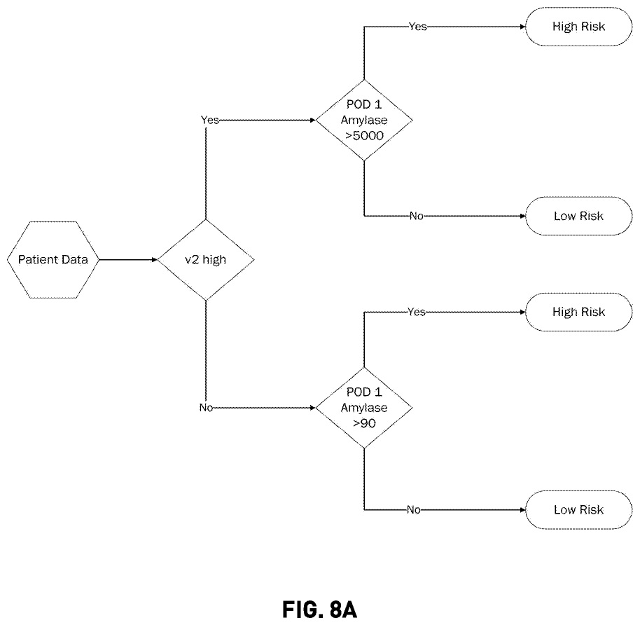

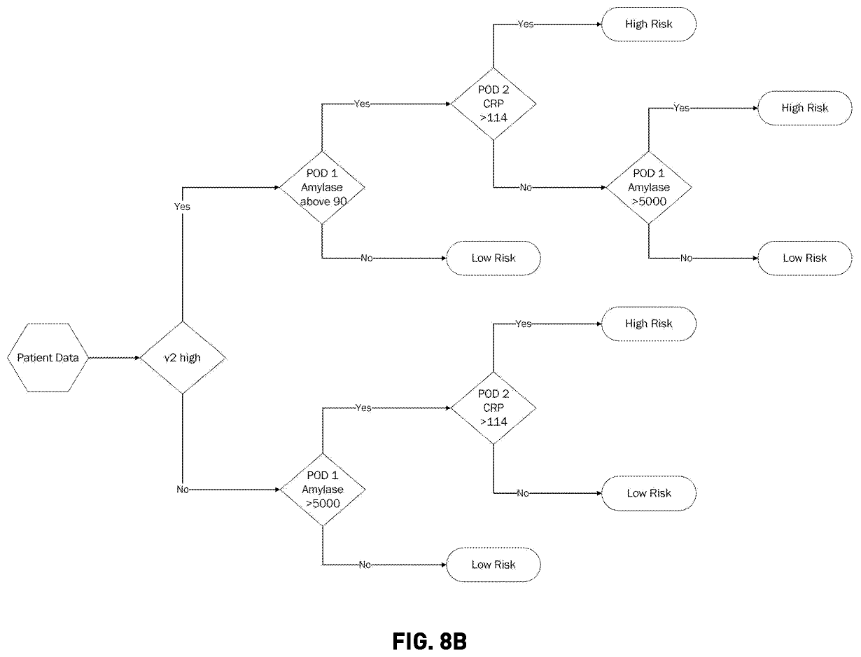

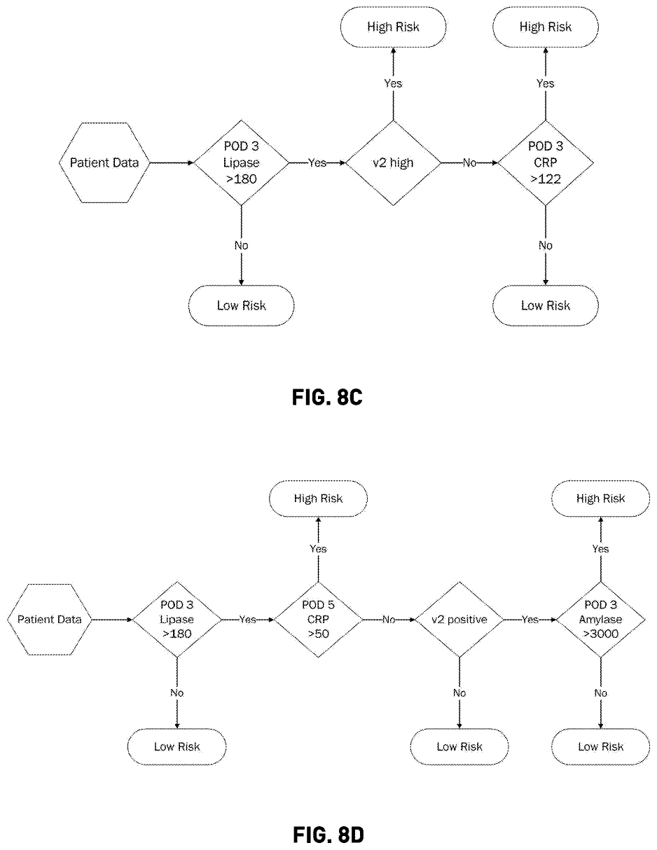

FIG. 8A illustrates a flowchart tested to ensure the simulated metrics would be similar to theoretical metrics. Given this flowchart, simulated performance metrics as shown in Table 6 were determined using the probability values relating to each path similar to the method described in paragraphs [0095] to [0097], wherein:
Sensitivity=P(High|BC)=P(Path1∪Path4|BC)=P(A_{high}∪(A_{low}∩B_{high}∩C_{high}) BC)=0.42+(0.58×0.91×0.78)=0.83
Specificity=P(Low|NOA)=P(Path2∪Path3|NOA)=P((A_{low}∩B_{low})∪(A_{low}∩B_{high}∪C_{low})|NOA)=(0.927×0.41)+(0.927×0.59×0.73)=0.78

TABLE 6
Simulated Performance Metrics Applied to a Simulated Dataset.

| Metric | Sensitivity (%) | Specificity (%) |
|---|---|---|
| POD 3 amylase >3000 U/L | 42 | 92.7 |
| POD 3 CRP >122 mg/L | 91 | 41 |
| v2 high | 78 | 73 |
| Overall Tree | 83 | 78 |

Using the simulated datasets, a decision tree with balanced class weight was separately trained on each PODs dataset and the splits or thresholds that were decided by the tree were examined. Exemplary decision trees and gates in relation to each POD is described herein.

A POD 1 decision tree as illustrated in FIG. 8A is constrained that a patient cannot have amylase levels greater than 5000 U/L and less than 90 U/L. The biomarkers used in this tree are the following: POD 1 Amylase>5000 U/L, POD 1 Amylase>90, and v2 high, wherein the biomarkers are assumed to be independent. The performance metrics in relation to the POD 1 decision tree are as follows:

TABLE 7
Simulated Performance Metrics Based on a POD 1 Decision Tree.

| Metric | Sensitivity (%) | Specificity (%) |
|---|---|---|
| POD 1 amylase >5000 U/L | 52 | 87 |
| POD 1 amylase >90 U/L | 96 | 45 |
| v2 high | 78 | 73 |
| Overall Tree | 87 | 76 |

A POD 2 decision tree illustrated in FIG. 8B uses the following biomarkers: POD 1 Amylase>5000 U/L, POD 1 Amylase>90, v2 high, and POD 2 CRP>114 mg/L. The tree is constrained that a patient cannot have amylase levels than 5000 U/L and less than 90 U/L. The performance metrics in relation to the POD 2 decision tree are as follows:

TABLE 8
Simulated Performance Metrics Based on a POD 2 Decision Tree.

| Metric | Sensitivity (%) | Specificity (%) |
|---|---|---|
| POD 1 amylase >5000 U/L | 52 | 87 |
| POD 1 amylase >90 U/L | 96 | 45 |
| v2 high | 78 | 73 |
| POD 2 CRP >114 mg/L | 93 | 41 |
| Overall Tree | 84 | 84 |

A POD 3 decision tree is illustrated in FIG. 8C, which uses the following biomarkers: POD 3 Lipase>180 U/L, POD 3 Amylase>3000 U/L, v2 high, and POD 3 CRP>122 mg/L. This tree is constrained that a patient cannot have amylase levels above 3000 U/L and lipase levels below 180 U/L. In this case, the exact biomarkers used in earlier PODs were not included since it is challenging to make reasonable assumptions on their interactions with other similar biomarkers. For example, there may be some correlation between the biomarker metrics of amylase>5000 U/L on PODi and amylase>3000 on PODW for a given patient. If we assumed complete correlation, the aforementioned biomarkers listed in Table 7 and Table 8 have been excluded in this decision tree, and only the latest non-corrected biomarkers are included (e.g. POD 3 lipase and amylase instead of POD 1 amylase; POD 3 CRP instead of POD 2 CRP). The performance metrics in relation to the POD 3 decision tree are as follows:

TABLE 9
Simulated Performance Metrics Based on a POD 3 Decision Tree.

| Metric | Sensitivity (%) | Specificity (%) |
|---|---|---|
| POD 3 amylase >3000 U/L | 42 | 93 |
| POD 3 lipase >180 U/L | 96 | 73 |
| v2 high | 78 | 73 |
| POD 3 CRP >122 mg/L | 91 | 41 |
| Overall Tree | 94 | 81 |

As for POD 4, the only new biomarker is POD 4 CRIP, which has the same sensitivity as POD 3 CRP with 10% lower specificity. As such, no new model was applied in this case.

A POD 5 decision tree is illustrated in FIG. 8D, which uses the following biomarkers: POD 3 Lipase>180 U/L, POD 3 Amylase>3000 U/L, v2 high, and POD 5 CRP>50 mg/L. In this case, POD 5 CRP was used instead of POD 3 CRP as used in FIG. 8C given that these two biomarkers may have some correlation. The general reasoning is similar to that the POD 3 decision tree description. The performance metrics in relation to the POD 5 decision tree are as follows:

TABLE 10
Simulated Performance Metrics Based on a POD 5 Decision Tree.

| Metric | Sensitivity (%) | Specificity (%) |
|---|---|---|
| POD 3 amylase >3000 U/L | 42 | 93 |
| POD 3 lipase >180 U/L | 96 | 73 |
| v2 high | 78 | 73 |
| POD 5 CRP >50 mg/L | 94 | 58 |
| Overall Tree | 92 | 88 |

TABLE 11
Theoretic Performance Metrics on Each POD.

| | Theoretical | Theoretical | Balanced |
|---|---|---|---|
| Configuration | Sensitivity (%) | Specificity (%) | Accuracy |
| POD 1 | 87 | 76 | 81 |
| POD 2 | 84 | 84 | 84 |
| POD 3 | 94 | 81 | 87 |
| POD 4 | N/A | N/A | N/A |
| POD 5 | 92 | 88 | 90 |

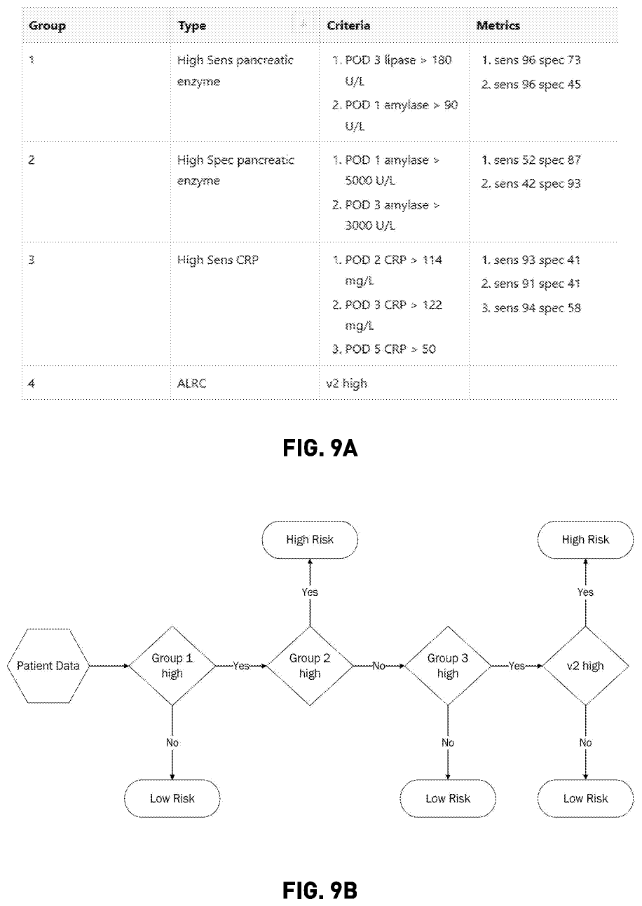

Although the theoretical performance of the decision tree in relation to each POD as summarized in Table 11 is promising, there may be some uncertainty regarding the interactions of the biomarkers and/or if the expected performance is reflective of true field performance. Deep decision trees may also involve more elaborate steps to handle missing data, which may add to the complexity of building risk prediction models by fitting decision trees with datasets to find optimal separations in data. As such, there is another method in which flowcharts are built that utilize gating biomarkers, among others. This method involves grouping gates by similarity and correlation and applying a set of rules. FIG. 9A is a table that outlines groupings applied to biomarker gates. Group 1 comprises pancreatic enzymes that have a high sensitivity. Group 2 comprises pancreatic enzymes that have a high specificity. Group 3 comprises c-reactive proteins that have a high sensitivity, while group 4 comprises ALRC model(s) that produce high predictions. A set of rules that may be applied when building flowcharts may comprise using one gate from each POD and using the latest available gate.

Given the groupings outlined in FIG. 9A and the aforementioned rules, an exemplary gating flowchart is used as shown in FIG. 9B. Group 1 is applied first because its pass-through metric (sensitivity) is highest at 96% for both tests as it minimizes error that propagates forward in the flowchart (minimal false negatives are funneled to low risk). Group 2 is applied second to compensate for the drop in sensitivity that would occur from group 1 being applied first. Lastly, group 3 is applied as a final gate. ALRC is applied last since it used the smallest sample size in its derivation, and its true performance is most likely to be different from expected performance.

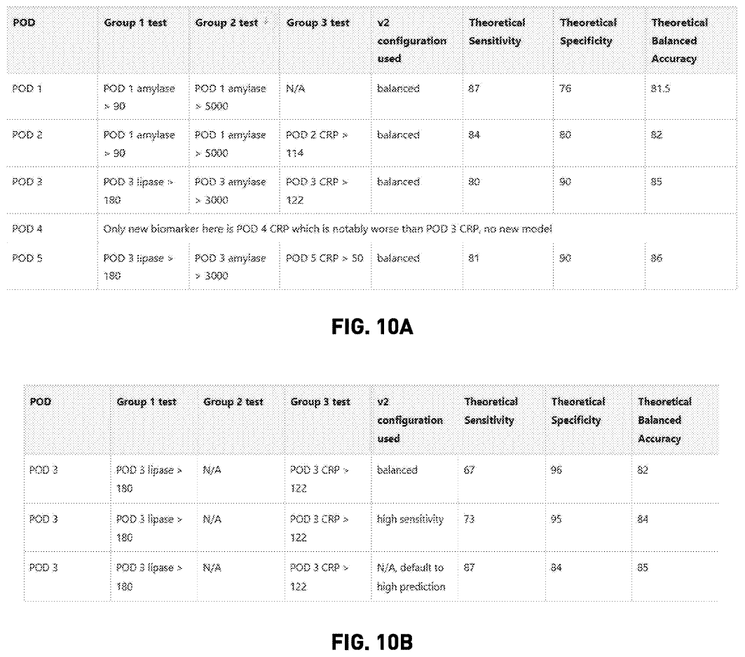

FIG. 10A is a table that outlines theoretical biomarker metrics for different scenarios in relation to various PODs. Using the aforementioned simplified workflow, missing data may be handled by excluding the gate that is associated with missing values. For example, sites are unlikely to measure both amylase and lipase. Therefore, there would likely be no group 2 test when a site does not measure amylase. This configuration, along with potential methods to improve performance are found in FIG. 10B. When there is no group 2 test, this will impact sensitivity. Therefore, one potential compensation is to use a higher sensitivity ALRC in this scenario, or to not use ALRC (and default its prediction to high).

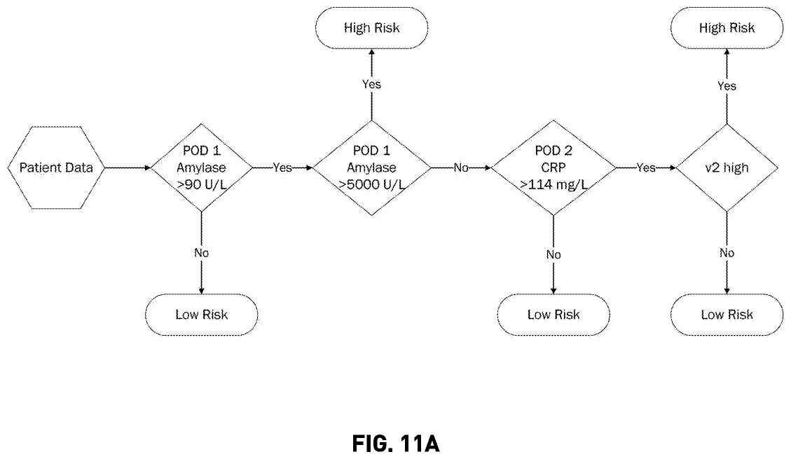

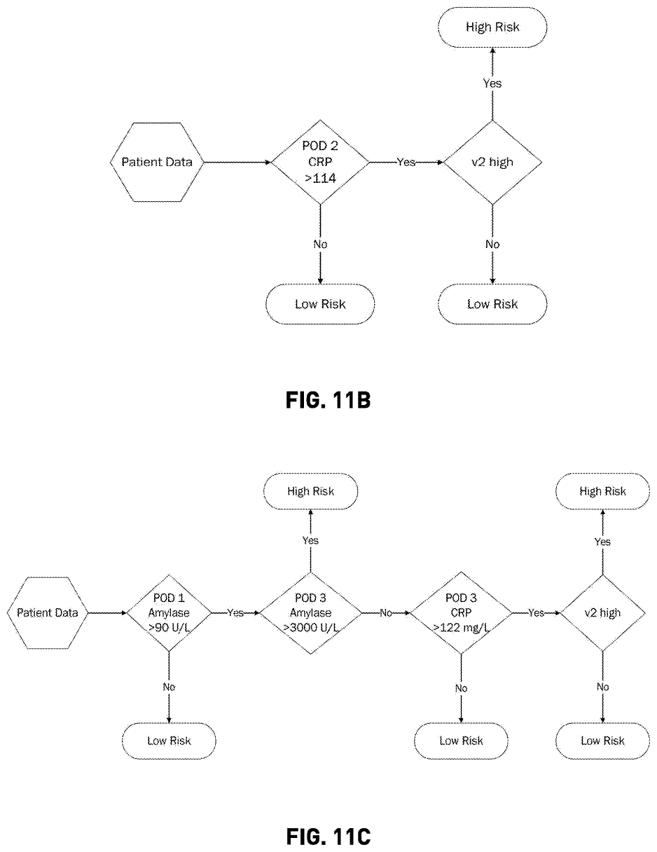

In some embodiments, the biomarker gates may expire after a certain amount of time (e.g., not use a gate from POD 1 on POD 4). If the expiration of gates applies to building the prediction model, the resulting risk scores may change if there is no replacement test. In this case, the risk score change is due to the expiration of a gate, and not due to any new information. For example, FIG. 11A shows a workflow that may be implemented using gates based on POD 1 amylase and POD 2 CRP. If tests are set to expire after three days and no new tests are taken, the workflow may become FIG. 11B on POD 4. In terms of the application of this workflow, a patient may have a POD 1 amylase>5000 U/L, causing them to be at a high risk of post-operative complications. After 4 days, the patient may no longer have that test taken into account, causing them to re-enter the flowchart and possibly be at a lower risk.

In some embodiments, the biomarker gates may be replaced when an alternative from the same group of tests is available. For example, if on POD 2, POD 1 amylase (containing a group 1 and group 2 test) and POD 2 CRP (containing a group 3 test) are available, the workflow shown in FIG. 11A may be implemented. FIG. 11C may then be implemented if on POD 4, the following biomarkers are available: POD 1 amylase (group 1+group 2), POD 2 CRP (group 3), POD 3 CRP (group 3), and POD 3 amylase (group 2).

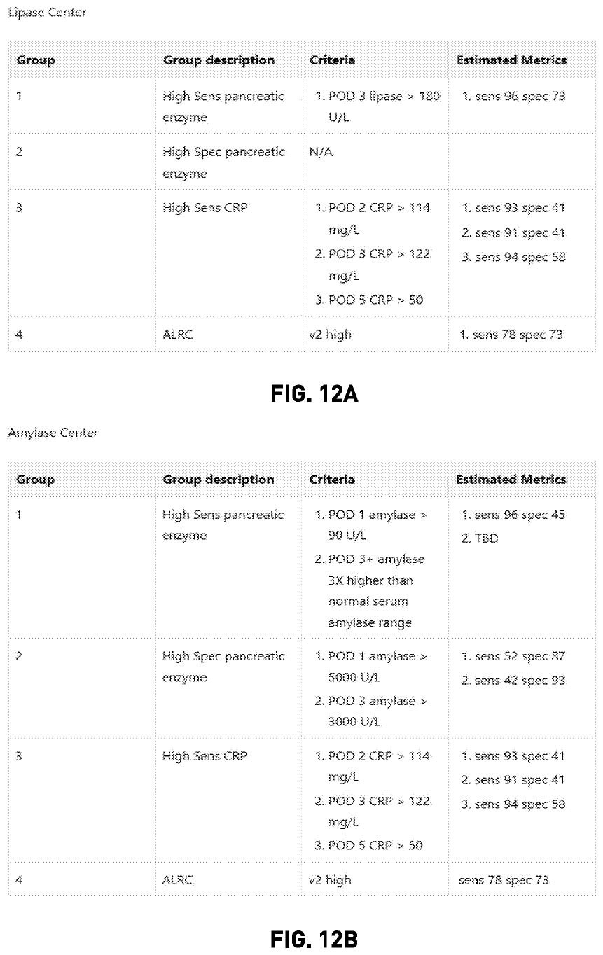

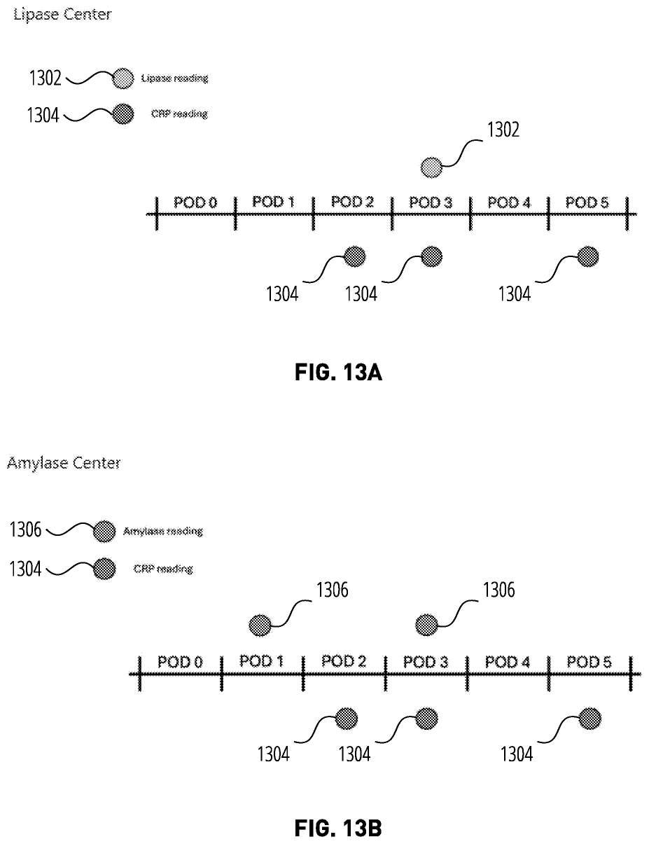

In some embodiments, one or more rules are applied to build an implementation of the post-operative risk prediction model using gating mechanisms. These rules may comprise (i) using the latest available test from each group as shown in FIG. 12A and FIG. 12B (e.g., if group 1 only has a test on POD 1, the test will still be used on POD 4 even if no replacement test is available), (ii) assuming the center may only have lipase or amylase, separating the workflows, and (iii) utilizing the standard definition of pancreatic fistula as a gate for POD 3 and later. Given the separation of amylase and lipase workflows, a plurality of potential workflows may apply. Graphical representations of the different times to apply the tests using the rules in terms of lipase and amylase are illustrated in FIG. 13A and FIG. 13B, respectively. In FIG. 13A, a single lipase reading 1302 is shown to be made in POD 3, while CRP readings 1304 are shown to be made in POD 2, POD 3 and POD 5. In FIG. 13B, amylase readings 1306 are shown to be made in POD 1 and POD 3, while CRP readings 1304 are shown to be made in POD 2, POD 3 and POD 5.

In some embodiments, the workflows and frameworks described herein may be used to chain together any type of decisions with known sensitivity and specificity values that may be unrelated to the use of biomarkers in risk prediction models.

In some embodiments, the post-operative risk prediction models built using the method and system described herein may be used to mitigate post-operative risk for patients. Exemplary mitigation measures may comprise modifying surgical plans (including the type of procedures, timing, duration, medications), effecting prevention procedures, and maintaining close surveillance of patients after surgery who are predicted to have a higher risk of complications. The post-operative risk prediction models may also be used to identify patients who should be given diagnostic tests (e.g., prompting a CT scan to test anastomotic leak after pancreatic or colorectal surgery) and/or identify patients unlikely to encounter post-operative complications who can be discharged from a healthcare facility at an earlier time than initially expected. Generally, the healthcare risk prediction framework described herein is configured to identify and/or prioritize medical procedures that diagnose, treat or prevent one or more patient outcomes (e.g., post-operative complications).

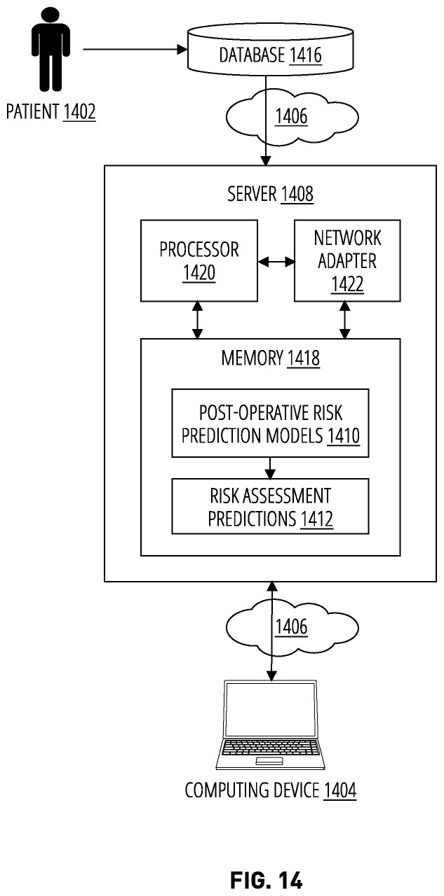

FIG. 14 is a schematic diagram of a computing system used to predict patient outcomes based on healthcare risk prediction models using gating mechanisms. According to an embodiment of the present disclosure, the system includes datasets in a database 1416 based on information from one or more patients 1406. This database may comprise scientific literature and simulated datasets, among others. The database 1416 is connected to a server 1408 through a cloud computing network 1406, wherein the connection between the database 1416 and the server 1408 is facilitated by a network adapter 1422. When a command is entered on a computing device 1404 to determine risk prediction of one or more patient outcomes for a given patient 1402, the computing device 1404 communicates with the server 1408 through a network 1406 to execute program instructions stored in a memory 1418 using a processor 1420. The memory 1418 is configured to store the healthcare risk prediction models 1410 and risk assessment predictions 1412. The program instructions within the memory allow the processor to run the healthcare prediction models 1410 to generate risk assessment predictions 1412.

The present disclosure includes systems having processors to provide various functionality to process information, and to determine results based on inputs. Generally, the processing may be achieved with a combination of hardware and software elements. The hardware aspects may include combinations of operatively coupled hardware components including microprocessors, logical circuitry, communication/networking ports, digital filters, memory, or logical circuitry. The processors may be adapted to perform operations specified by a computer-executable code, which may be stored on a non-transitory computer readable medium.

The steps of the methods described herein may be achieved via an appropriate programmable processing device or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments discussed above and appreciated by those skilled in the computer and software arts. Appropriate software can be readily prepared by programmers of ordinary skill based on the teachings of the exemplary embodiments, as is appreciated by those skilled in the software arts. In addition, the devices and subsystems of the exemplary embodiments can be implemented by the preparation of application-specific integrated circuits, as is appreciated by those skilled in the electrical arts. Thus, the exemplary embodiments are not limited to any specific combination of hardware circuitry and/or software.

Stored on any one or a combination of computer readable media or non-transitory computer readable media, the exemplary embodiments of the present invention may include software for controlling the devices and subsystems of the exemplary embodiments, for processing data and signals, for enabling the devices and subsystems of the exemplary embodiments to interact with a human user or the like. Such software can include, but is not limited to, device drivers, firmware, operating systems, development tools, applications software, and the like. Such computer-readable media further can include the computer program product of an embodiment of the present invention for preforming all or a portion (if processing is distributed) of the processing performed in implementations. Computer code devices of the exemplary embodiments of the present invention can include any suitable interpretable or executable code mechanism, including but not limited to scripts, interpretable programs, dynamic link libraries (DLLs), complete executable programs and the like.

Common forms of computer-readable media may include, for example, magnetic disks, flash memory, RAM, a PROM, an EPROM, a FLASH-EPROM, or any other suitable memory chip or medium from which a computer or processor can read.

Embodiments of the subject matter described in this specification can be implemented in a computing system that includes: a front end component (e.g., a computer having a graphical user interface or a Web browser though which a user can interact with an implementation of the subject matter described herein); or a midware component (e.g., an application server); or a back end component (e.g., a data server); or any combination of one or more such back end, middleware, or front end components. The components of the system can be interconnected by any form or medium of digital data communication (e.g., a communication network). Non-limiting examples of communication networks include a local area network (“LAN”) and a wide area network (“WAN”).

Processor may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system can include, in addition to hardware, code that creates an execution environment for the computer program in question, enabling it to utilize the built healthcare risk prediction models using gating mechanisms e.g., code that constitutes processor firmware, a protocol stack, a database management system, an operating system, or a combination of one or more of them stored in an included memory, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus for storing information and instructions to be executed by processor. The processor and the memory can be supplemented by, or incorporated in, special purpose logic circuitry. The sensor device may have its own memory for storing data. The memory may be in a separate computer system. The memory may comprise cloud data storage.

The instructions may be stored in the memory and implemented in one or more computer program products, i.e., one or more modules of computer program instructions encoded on a computer readable medium for execution by, or to control the operation of, the computer system, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory may also be used for storing temporary variable or other intermediate information during execution of instructions to be executed by processor.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Information as herein shown and described in detail is fully capable of attaining the above-described object of the present disclosure, the presently preferred embodiment of the present disclosure, and is, thus, representative of the subject matter which is broadly contemplated by the present disclosure.

What is claimed is:

1. A computer-implemented method for building a healthcare risk prediction framework, the method comprising:
receiving, at a server through a network, one or more biomarkers of one or more patients and one or more existing risk prediction models;
selecting, using a processor, a set of predictors based on a predetermined threshold of the one or more biomarkers or existing risk prediction models, wherein the set of predictors comprises the one or more biomarkers, the one or more existing risk prediction models or a combination thereof,
estimating, using the processor, performance of each element in the set of predictors in terms of specificity and sensitivity;
combining, using the processor, the set of predictors in a flowchart or a decision tree to generate the healthcare risk prediction framework;
calculating, using the processor, a performance estimate of each path in the flowchart or the decision tree;
calculating, using the processor, the performance estimate of the flowchart or the decision tree;
determining a risk prediction of one or more patient outcomes based on the flowchart or the decision tree having an optimal performance estimate; and
effecting one or more medical procedures to be identified or prioritized based at least in part on the risk prediction, wherein the one or more medical procedures diagnoses, treats or prevents the one or more patient outcomes.

2. The computer-implemented method of claim 1, further comprising optimizing the combination of the set of predictors in the flowchart or the decision tree using predefined metrics.

3. The computer-implemented method of claim 2, wherein the predefined metrics comprise specificity, sensitivity, accuracy, balanced accuracy, likelihood ratios, predictive values, and F1 scores.

4. The computer-implemented method of claim 1, wherein the performance of each element in the set of predictors is estimated by using experimentation or pooled meta-analysis techniques.

5. The computer-implemented method of claim 1, wherein the combining the set of predictors is based on balancing specificity and sensitivity to minimize false negatives and false positives.

6. The computer-implemented method of claim 1, wherein the flowchart or the decision tree is configured to distinguish cases with a high likelihood of complications and low likelihood of complications.

7. The computer-implemented method of claim 1, wherein the performance estimate of each path in the flowchart or the decision tree is calculated using simulations or probability theory.

8. The computer-implemented method of claim 1, further comprising estimating bounds of expected performance based on dependence between each element in the set of predictors using Fréchet inequalities or simulation techniques.

9. The computer-implemented method of claim 1, wherein the predetermined threshold is determined based on consistent reported data from scientific literature or using simulation techniques.

10. The computer-implemented method of claim 1, wherein the one or more biomarkers comprise at least one of: c-reactive proteins, lipase, amylase, white blood count, procalcitonin, lactate, bilirubin, or neutrophil-lymphocyte ratios.

11. A system for building a healthcare risk prediction framework, the system comprising:
a computing device connected to a server through a network, wherein the server comprises:
a memory; and
a processor coupled to the memory comprising program instructions, wherein the program instructions are executable by the processor to perform operations comprising:
receiving, at a server through a network, one or more biomarkers of one or more patients and one or more existing risk prediction models;
selecting, using a processor, a set of predictors based on a predetermined threshold of the one or more biomarkers or existing risk prediction models, wherein the set of predictors comprises the one or more biomarkers, the one or more existing risk prediction models or a combination thereof,
estimating, using the processor, performance of each element in the set of predictors in terms of specificity and sensitivity;
combining, using the processor, the set of predictors in a flowchart or a decision tree to generate the healthcare risk prediction framework; and
calculating, using the processor, a performance estimate of each path in the flowchart or the decision tree;
calculating, using the processor, the performance estimate of the flowchart or the decision tree;
determining a risk prediction of one or more patient outcomes based on the flowchart or the decision tree having an optimal performance estimate; and
effecting one or more medical procedures to be identified or prioritized based at least in part on the risk prediction, wherein the one or more medical procedures diagnoses, treats or prevents the one or more patient outcomes.

12. The system of claim 11, wherein the operations further comprise optimizing the combination of the set of predictors in the flowchart or the decision tree using predefined metrics.

13. The system of claim 12, wherein the predefined metrics comprise specificity, sensitivity, accuracy, balanced accuracy, likelihood ratios, predictive values, and F1 scores.

14. The system of claim 11, the performance of each element in the set of predictors is estimated by using experimentation or pooled meta-analysis techniques.

15. The system of claim 11, wherein the combining the set of predictors is based on balancing specificity and sensitivity to minimize false negatives and false positives.

16. The system of claim 11, wherein the flowchart or the decision tree is configured to distinguish cases with a high likelihood of complications and low likelihood of complications.

17. The system of claim 11, wherein the performance estimate of each path in the flowchart or the decision tree is calculated using simulations or probability theory.

18. The system of claim 11, wherein the operations further comprise estimating bounds of expected performance based on dependence between each element in the set of predictors using Fréchet inequalities or simulation techniques.

19. The system of claim 11, wherein the predetermined threshold is determined based on consistent reported data from scientific literature or using simulation techniques.

20. The system of claim 11, wherein the one or more biomarkers comprise at least one of: c-reactive proteins, lipase, amylase, white blood count, procalcitonin, lactate, bilirubin, or neutrophil-lymphocyte ratios.
